(19) United States
(12) Patent Application Publication
(10) Pub. No.: US 2010/0030247 A1
(43) Pub. Date: Feb. 4, 2010

(54) VARICOSE VEIN REMOVAL DEVICE

(75) Inventors: Valery Pikus, Ashdod (IL); Yacov Schwartz, Ashdod (IL); Yigal Schwartz, Ashdod (IL); Boris Gliner, Rechovot (IL)

Correspondence Address: MARTIN D. MOYNIHAN d/b/a PRTSI, INC., P.O. BOX 16446, ARLINGTON, VA, 22215, US

(73) Assignee: SIMEDIQ Medical Equipment Ltd., Ashdod (IL)

(21) Appl. No.: 12/525,091

(22) PCT Filed: Jan. 29, 2008
(86) PCT No.: PCT/IL08/00129

§ 371 Date: Jul. 30, 2009

Related U.S. Application Data
(60) Provisional application No. 60/898,714, filed on Feb. 1, 2007.

(51) Int. Cl. A61B 17/22; A61B 17/32

(52) U.S. Cl. 606/159; 606/167

(57) ABSTRACT

Disclosed is an instrument for treating varicose veins, the instrument comprising: an elongate shaft having a longitudinal axis and configured to be manually moved in a forward direction and a rearward direction through a portion of soft tissue. The instrument further comprises at least two teeth projecting substantially on a first plane from the elongate shaft, the at least two teeth respectively having a forward facing surface and a rearward facing surface. The forward facing surface is angularly configured to allow substantially atraumatic passage through a tissue portion when the instrument is moved in a forward direction. The rearward facing surface is angularly configured to cause substantially traumatic passage through a tissue portion when the instrument is moved in a rearward direction.

### FIELD AND BACKGROUND OF THE INVENTION

The present invention relates to devices for treating varicose veins in general, and in particular, to a surgical instrument for removing varicose veins.

Varicose veins are a condition of the superficial veins of the lower extremities in which one or more valves within the veins have ceased to function efficiently, resulting in blood pooling in the legs. The resultant build up of pressure causes the veins to become abnormally distorted and prominent, causing heaviness, stiffness and fatigue in the legs as well as bluish/purple bulges that are undesirable from a cosmetic point of view. Untreated varicose veins may cause severe pain, eczema and leg ulcers.

Some 20% of women in the general population suffer from varicose veins. In an estimated 5-7% of sufferers an operation is required in order to remove the damaged veins and encourage blood flow through alternative routes. In other varicose vein sufferers, surgery is required to remove the unsightly veins to obtain cosmetic improvement.

In the conventional techniques used for varicose vein removal, an incision is made over a portion of a varicose vein and the vein is incised. The cut end of the vein is grasped and stretched with a surgical clamp and up to five centimeters of the vein is dissected, torn from the remaining vein portion and pulled out of the incision.

To remove multiple varicose veins, as many as ten to fifteen incisions are typically required. The many incisions present a risk of damage to nerves and small arteries. Additionally, the many incisions present a risk of becoming inflamed, infected and/or form unattractive scars.

U.S. Pat. No. 6,436,116 (Spitz et al), the content of which is incorporated by reference as if fully set forth herein, teaches a surgical cutting instrument including a light source in order to pinpoint the location of a varicose vein; a complex device to manufacture and utilize.

U.S. Pat. No. 5,792,168 (Seval), the content of which is incorporated by reference as if fully set forth herein, teaches a cylindrical sleeve enclosing a sliding shaft that are manipulated together to grip a vein portion; a device that fails to reduce the number of incisions required during an operation.

World IPO Patent WO9421177A1 (Christoffersson, et al), the content of which is incorporated by reference as if fully set forth herein, teaches a cone of barbs that is passed through the vein to grasp the vein lumen, but again, fails to significantly reducing the high number of required incisions.

World IPO Patent Application WO2005107371 and Australia Patent Application AU2005239907 (Gliner et al), the content of which are incorporated by reference as if fully set forth herein, teach retractable vein engaging elements attached to a retraction mechanism and set in a cylindrical hollow shaft; the diameter of the hollow shaft requiring relatively large tissue incisions to access the veins.

Accordingly, there is a need for a surgical device configured for cutting and removing varicose veins through a few small incisions.

### SUMMARY OF THE INVENTION

The present embodiments successfully address the shortcomings of present known varicose vein devices and procedures by providing a device configured for removing multiple and/or elongate portions of varicose veins from a few incisions.

In embodiments, the device of the present invention provides a shaft having multiple teeth having forward angled and rearward angled surfaces. The forward angled surface is sloped to minimize tissue damage as the shaft is pushed forward through tissue. The rearward angled surface is configured to maximize damage to tissue, specifically varicose veins, as the shaft is pulled rearward through tissue.

Additionally, the instrument is configured to wrap the vein around the shaft as the shaft is rotated, and sever and remove substantially large portions of veins as the shaft is pulled rearward, thereby requiring a minimum of incisions to remove the varicose veins.

According to an aspect of the invention, there is provided an instrument for treating varicose veins, the instrument comprising: an elongate shaft having a longitudinal axis and configured to be manually moved in a forward direction and a rearward direction through a portion of soft tissue.

The instrument further comprises at least two teeth projecting substantially on a first plane from the elongate shaft, the at least two teeth respectively having a forward facing surface and a rearward facing surface. The forward facing surface is angularly configured to allow substantially atraumatic passage through a tissue portion when the instrument is moved in a forward direction. The rearward facing surface is angularly configured to cause substantially traumatic passage through a tissue portion when the instrument is moved in a rearward direction.

In embodiments, the forward facing surface forms an angle of between 25-35 degrees with a longitudinal shaft axis.

In embodiments, the forward facing surface forms an angle of about 30 degrees with a longitudinal shaft axis.

In embodiments, the rearward facing surface and forward facing surface form an angle of between 50 and 60 degrees.

In embodiments, the rearward facing surface and forward facing surface form an angle of about 53 degrees.

In embodiments, the shaft has a length of between 80.0 and 120.0 millimeters.

In embodiments, the shaft has a length of about 100.0 millimeters.

In embodiments, the shaft has a thickness of between 1.0 and 3.0 millimeters.

In embodiments, the shaft has a thickness of about 1.6 millimeters.

In embodiments, the shaft has a height of between 3.0 and 6.0 millimeters.

In embodiments, the shaft has a height of between 3.5 and 4.6 millimeters.

In embodiments, the at least two teeth each have a length of between 2.0 and 3.0 millimeters.

In embodiments, the at least two teeth each have a length of about 2.3 millimeters.

In embodiments the at least two teeth each have a height of between 1.0 and 2.0 millimeters.

In embodiments, the at least two teeth each have a height of about 1.4 millimeters.

In embodiments, the instrument includes a handle.

In embodiments, the handle has a length of between about 80.0 and 120.0 millimeters.

In embodiments, the handle has a length of about 100.0 millimeters.

In embodiments, the handle has a height of between 10.0 and 14.0 millimeters.

In embodiments, the handle has a height of about 12.0 millimeters.

In embodiments, the instrument further comprises a substantially tapered tip located at an end of the shaft, the tip being configured for allowing introduction of the instrument into an incision through a skin layer.

In embodiments, the instrument further comprises a substantially pointed tip located at an end of the shaft, the tip being configured for creating an incision through a skin layer.

In embodiments, the shaft is fixedly attached to a handle.

In embodiments, the shaft is removably attached to a handle.

In embodiments, the shaft is substantially rigid.

In embodiments, the instrument includes at least two substantially planar teeth projecting on a second plane from the elongate shaft.

In embodiments, the first plane is substantially planar with the second plane.

In embodiments, the instrument includes at least two substantially planar teeth projecting on a third plane from the elongate shaft.

In embodiments, the instrument includes at least two substantially planar teeth projecting on a fourth plane from the elongate shaft.

In embodiments, the at least two teeth are configured to provide at least one of: puncturing a vein, engaging a vein, tearing at least a portion of a vein, ensnaring at least a portion of a vein, wrapping at least a portion of a vein around the shaft while rotating the shaft along a longitudinal axis of the shaft, and severing a first portion of a vein from a second portion of a vein.

According to another aspect of the invention, there is provided a method of treating superficial varicose veins in a body region, the method comprising: forming an incision in a skin layer adjacent to the body region, inserting, through the incision an instrument comprising a shaft having at least two teeth configured for engaging a vein such that each of the two teeth having a forward facing surface forming an angle of between 25-35 degrees with a longitudinal shaft axis and a rearward facing surface forming an of between 50 and 60 degrees with the forward facing surface.

The method further comprises rotating the shaft along a longitudinal axis of the shaft, wrapping at least a portion of at least one first vein around the shaft, removing the instrument, thereby treating the superficial varicose veins in the body region.

In embodiments, the instrument comprises a substantially sharp tip located at an end of the shaft and the incision in the skin layer is formed using the substantially sharp tip.

In embodiments, the treating additionally comprises at least one of: sweeping the shaft parallel to a surface of the skin layer, ensnaring at least a portion of at least one first vein, and pulling at least a portion of at least one first vein.

In embodiments, the treating additionally comprises at least one of: puncturing, engaging, tearing, and removing at least a portion of at least one first vein through the incision.

In embodiments, the method includes severing a first portion of at least one vein from a second portion of at least one vein and leaving the vein in a portion of tissue.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

### BRIEF DESCRIPTION OF THE DRAWINGS

The invention of a device for removing multiple elongate portions of varicose veins from a single incision is herein described, by way of example only, with reference to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a typical varicose vein engaging instrument, according to embodiments of the present invention;

FIG. 2 shows details of the vein engaging instrument shown in FIG. 1, according to embodiments of the present invention;

FIGS. 3A-3C show a typical varicose vein removal operation using the vein engaging instrument shown in FIG. 1, according to embodiments of the present invention; and

FIGS. 4-5 show alternative embodiments of the vein engaging instrument shown in FIG. 1, according to embodiments of the present invention.

### DESCRIPTION OF THE PREFERRED EMBODIMENTS

The present invention relates to a device for removing multiple elongate portions of varicose veins from a leg. The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Referring now to the drawings:

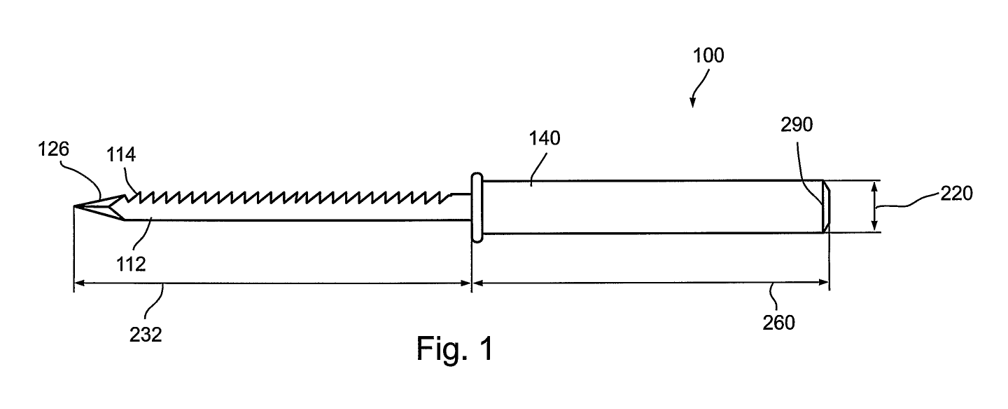

FIG. 1 shows a varicose vein-engaging instrument 100 having a handle 140 and an elongate serrated shaft 112 projecting therefrom. Each vein-engaging too 114, alternatively referred to as tooth 114, project radially outward from shaft 112, substantially on a single plane.

Shaft 112 is optionally flattened like a knife blade, or curved to form, for example, a cylindrical shape or any other configuration that allows passage through tissue. Shaft 112 and handle 140 are typically fabricated from any appropriate biocompatible material including, for example, polymers, metals or any combination thereof.

Handle 140 typically has a length 260 of between about 80.0 and 120.0 millimeters though in alternative embodiments, length 260 is about 100.0 millimeters.

Handle 140 typically has a height 220 (or diameter) of between about 10.0 and 14.0 millimeters though in alternative embodiments, height 220 is about 12.0 millimeters.

Shaft 112 typically has a length 232 of between 80.0 and 120.0 millimeters though in alternative embodiments length 232 is about 100.0 millimeters.

As shown handle 140 has a flat end 290 and shaft 112 includes a tissue and skin-cutting tip 126 that is configured to incise the skin.

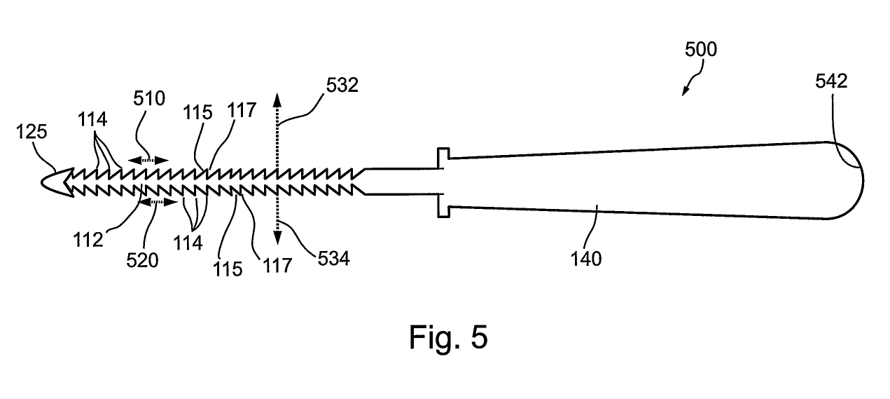

In an alternative embodiment, seen in FIG. 5, shaft 112 features a tapered tip 125 that typically requires a separate skin incision with a scalpel. After forming the incision, tapered tip 125 is inserted into the incision and, by pressing instrument 500 forward, separates tissue.

In embodiments, handle 140 features a curved end 542; the many configurations for handle 140 being well known to those familiar with the art. In embodiments, the shaft has a length of about 100.0 millimeters.

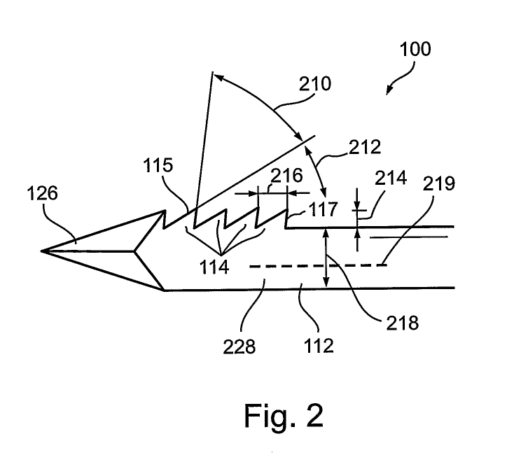

As seen in FIG. 2, shaft 112 typically has a thickness 228 of between 1.0 and 3.0 millimeters, though in embodiments thickness 228 is about 1.6 millimeters. Further, shaft 112 typically has a height 218 of between 3.0 and 6.0 millimeters, though in alternative embodiments, height 218 is between 3.5 and 4.6 millimeters.

Vein engaging teeth 114 typically have a forward facing surface 115 and a rearward facing surface 117. Tooth 114 typically has a length 216 of between 2.0 and 3.0 millimeters though in alternative embodiments length 216 is about 2.3 millimeters. Additionally, tooth 114 typically has a height 214 of between 1.0 and 2.0 millimeters though in alternative embodiments height 214 is about 1.4 millimeters.

In embodiments, forward facing surface 115 typically forms a forward angle 212 with a longitudinal axis 219 of between about 25-35 degrees. In alternative embodiments forward angle 212 is about 30 degrees or any forward angle 212 that facilitates separation of tissue with substantially minimal damage when shaft 112 is pushed in a forward direction through tissue.

Rearward facing surface 117 typically forms a rearward angle 210 with forward facing surface 115 of between 50 and 60 degrees. In alternative embodiments rearward angle 210 is about 53 degrees or any rearward angle 210 that facilitates snaring, pulling and/or incising a varicose vein when shaft 112 is pulled in a rearward direction through tissue.

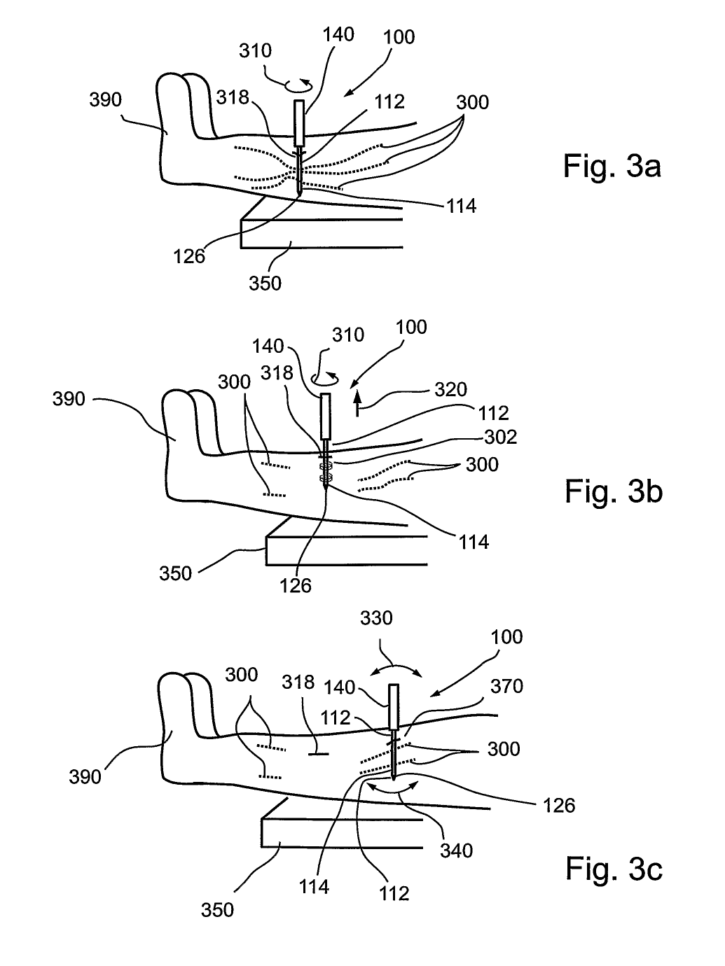

As seen in FIG. 3A, during a typical operation, a leg 390 of a subject reclining on a surgical table 350 is incised with sharp tip 126, thereby forming incision 318.

Instrument 100 is advanced subcutaneously transverse to and proximate to varicose veins 300. In embodiments, instrument 100 is rotated around shaft 112 in a circular direction 310.

As seen in FIG. 3B, rotation of instrument 100 in direction 310 has caused varicose vein portions 302 to become enwrapped around shaft 112, thereby causing vein portions 302 to sever from remaining vein portions 300. The ability to rotate instrument 100 longitudinally around the axis of shaft 112, allows substantially long lengths of vein portions 302 to wrap around shaft 112. Instrument 100 is then pulled in a backward direction 320 out of incision 318 and vein portions 302 are removed from shaft 112.

As used herein, forward movement refers to pressing instrument 112 forward into leg 390 while rearward movement refers to pulling instrument 112 rearward and out of leg 390.

As seen in FIG. 3C, sharp tip 126 has been used to create a second incision 370 and instrument 100 is proximate to varicose veins 300 in the upper area of leg 390. In embodiments, handle 140 is swept in backward and forward directions 330 substantially along a single plane. With incision 370 being a pivot point, shaft 112 sweeps forward and backward in directions 340, thereby ensnaring varicose veins 300 in an area of tissue along the entire length of shaft 112. Long shaft 112 and sweeping motions maximize the number and length of varicose veins 300 that are removed from a single incision 370.

The remaining portions of varicose veins 300 in the lower portion of leg 390 are small and typically collapse following the operation, thereby removing the necessity for further incisions. Alternatively, additional incisions are made to remove varicose veins 300 in the lower portion of leg 390.

Bleeding from veins 300 typically stops without assistance or by applying pressure on incision 318. In cases where bleeding continues, the ends of veins 300 are optionally tied with suture, using techniques that are well known in the art.

It will be appreciated that vein engaging instrument 100 pivots to multiple directions through incisions 318 and 370 in order to engage the desired portions of veins 300 along the entire length of shaft 112. Using instrument 100, few incisions 318 and 370 are required for the surgeon to remove multiple portions and long lengths of veins 300, thereby reducing the risk for hematoma, inflammation, infection, and scarring.

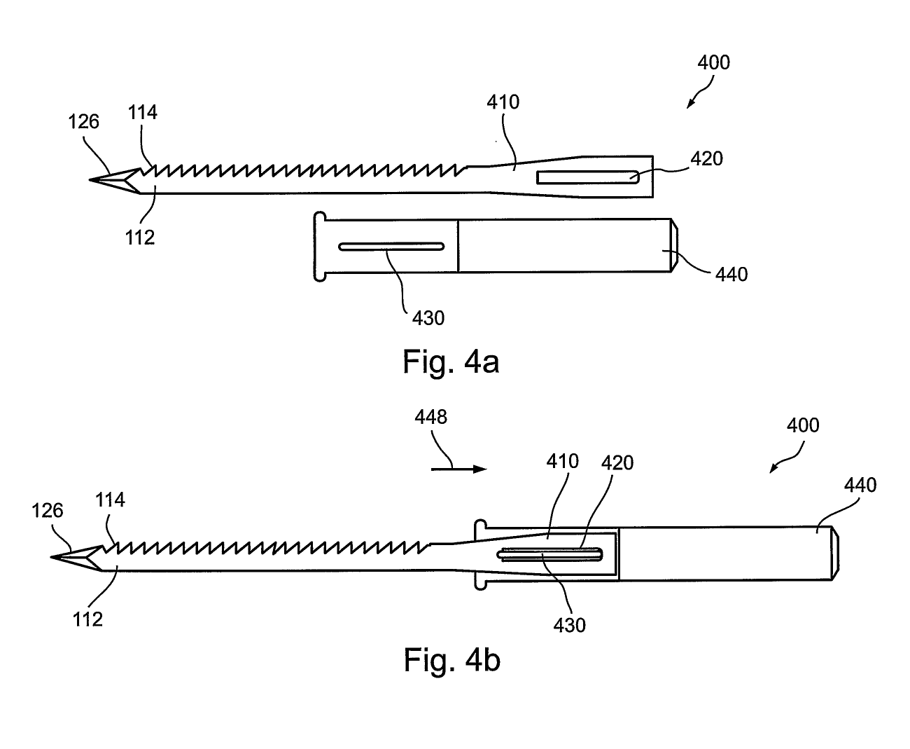

In embodiments, as seen in FIGS. 4A and 4B, shaft 112 is disposable and attaches to a handle 440. In non-limiting embodiments, handle 440 includes an offset 430 and shaft 112 includes a side base 410 having an offset receptacle 420.

Attachment of shaft 112 to handle 440 entails aligning receptacle 420 with offset 430 and sliding backward in a direction 448 so that offset 430 secures shaft 112 to handle 440. The many options for connections between shaft 112 and handle 440 are well known to those familiar with the art.

In embodiments, as seen in FIG. 5, shaft 112 includes a first tooth set 510 projecting in a first direction and a second tooth set 520 projecting in a second direction.

Optionally, the first direction 532 is 180 degrees to the second direction 534 and first tooth set 510 is planar with second tooth set 520. Alternatively, first tooth set 510 forms an angle of between about 90 degrees and 180 degrees with second tooth set 520.

In still other embodiments, shaft 112 includes three or even four tooth sets 510 positioned, for example, radially around shaft 112. The many configurations of tooth sets 510 are well known to those familiar with the art.

The Examples section below provides results obtained using a vein engaging instrument of a previous proposal in treating varicose veins, and the discussion indicates where the present embodiments are advantageous.

### EXAMPLES

Reference is now made to examples which, together with the above description, illustrate the use of vein engaging instrument that was filed by the inventors in a prior patent, comprising retractable vein-engaging teeth and a shaft having a diameter of 4.0 millimeters.

The present embodiments, using fixed position vein engaging teeth, are expected to have better results, for example, due to the smaller diameter shaft. The smaller shaft allows reduction of the size of the incision through which the instrument is introduced into the tissue, thereby reducing external bleeding and producing a cosmetically pleasing result in a shorter period of time.

Using a vein engaging instrument having retractable teeth and a 4.0 millimeters shaft diameter, trial procedures were performed on live patients and cadavers having varicose veins in their lower extremities. The live patient age ranged between 25 and 65 years old and the deceased age of the cadavers ranged between 30 and 85 years old.

In all cases where varicose veins were identified, the veins were successfully engaged, torn and removed. Inspection of the vein engaging instrument indicated that no tissue other than vein and subcutaneous adipose tissue were substantially damaged by the instrument. For example, no remains of muscle or nerve tissue were observed on the instrument.

In each procedure two to four incisions were made in skin regions adjacent to the varicose veins and the total procedure time was between 5 and 10 minutes. In all cases there no internal bleeding was observed and only substantially small hematomas were formed. External bleeding was stopped using applied pressure for 1-3 minutes.

Following surgery, all patients were immediately bandaged with standard elastic bandages and re-bandaged the following day. During the first 48 hours following surgery, pain-relief medication was prescribed, comprising a non-narcotic analgesic.

None of the patients complained of strong pain which could be indicative of under-skin hematomas or nerve damage; likely as a result of the small number of incisions and resultant reduced damage to the tissue. During a 7-10 day recovery period no complications were observed and all of the patients returned to normal life including work.

The results of these trials showed that the vein engaging instrument of the retractable tooth variety effectively treats varicose veins. It is considered that the vein-engaging teeth of the vein engaging instrument according to the present embodiments enable highly effective grasping and tearing of varicose veins even in cases where such veins are extended and entangled.

In addition, the above-described trials illustrated that the vein engaging instrument tested in the above example, enabled complete removal of all varicose veins through about 2-4 incisions.

Moreover, in instruments configured with a pointed tip at the end of the shaft, there was no need for making separate incisions as the tip easily penetrated the skin. Further, in using the pointed tip, there was no need for stitches following removal of the vein engaging instrument.

The clinical trials described above demonstrated that the vein engaging instrument tested is highly effective for treating varicose veins and achieves positive cosmetic results.

The vein engaging instrument of the present embodiments is an improvement thereon in that it enables to deal with entangled and twisted veins, and requires a smaller incision.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An instrument for treating varicose veins, said instrument comprising:
a) an elongate shaft having a longitudinal axis and configured to be manually moved in a forward direction and a rearward direction through a portion of soft tissue;
b) at least two teeth projecting substantially on a first plane from said elongate shaft, said at least two teeth respectively having a forward facing surface and a rearward facing surface:
i) said forward facing surface being angularly configured to allow substantially atraumatic passage through a tissue portion when said instrument is moved in a forward direction; and
ii) said rearward facing surface being angularly configured to engage at least one of the varicose veins when said instrument is moved in a rearward direction.

2. The instrument according to claim 1, wherein said forward facing surface forms an angle of between 25-35 degrees with a longitudinal shaft axis.

3. (canceled)

4. The instrument according to claim 1, wherein said rearward facing surface and forward facing surface form an angle of between 50 and 60 degrees.

5. (canceled)

6. The instrument according to claim 1, wherein said shaft has a length of between 80.0 and 120.0 millimeters.

7. (canceled)

8. The instrument according to claim 1, wherein said shaft has a thickness of between 1.0 and 3.0 millimeters.

9. (canceled)

10. The instrument according to claim 1, wherein said shaft has a height of between 3.0 and 6.0 millimeters.

11. (canceled)

12. The instrument according to claim 1, wherein said at least two teeth each have a length of between 2.0 and 3.0 millimeters.

13. (canceled)

14. The instrument according to claim 1, wherein said at least two teeth each have a height of between 1.0 and 2.0 millimeters.

15-20. (canceled)

21. The instrument according to claim 1, further comprising a substantially tapered tip located at an end of said shaft, said tip being configured for allowing introduction of said instrument into an incision through a skin layer.

22. The instrument according to claim 1, further comprising a substantially pointed tip located at an end of said shaft, said tip being configured for creating an incision through a skin layer.

23-24. (canceled)

25. The instrument according to claim 1, wherein said shaft is substantially rigid.

26. The instrument according to claim 1, including at least two substantially planar teeth projecting on a second plane from said elongate shaft.

27. The instrument according to claim 26, wherein said first plane is substantially planar with said second plane.

28. The instrument according to claim 26, including at least two substantially planar teeth projecting on a third plane from said elongate shaft.

29. The instrument according to claim 28, including at least two substantially planar teeth projecting on a fourth plane from said elongate shaft.

30. The instrument according to claim 1, wherein said at least two teeth are configured to provide at least one of:
a) puncturing a vein;
b) engaging a vein;
c) tearing at least a portion of a vein;
d) ensnaring at least a portion of a vein;
e) wrapping at least a portion of a vein around said shaft while rotating said shaft along a longitudinal axis of said shaft; and
f) severing a first portion of a vein from a second portion of a vein.

31-36. (canceled)

37. A method of treating superficial varicose veins in a body region, the method comprising:
(a) forming an incision in a skin layer adjacent to the body region;
(b) inserting, through said incision an instrument comprising a shaft having at least two teeth configured for engaging a vein such that each of said two teeth having a forward facing surface forming an angle of between 25-35 degrees with a longitudinal shaft axis and a rearward facing surface forming an of between 50 and 60 degrees with the forward facing surface;
(c) rotating said shaft along a longitudinal axis of said shaft;
(d) wrapping at least a portion of at least one first vein around said shaft;
(e) removing said instrument, thereby treating the superficial varicose veins in the body region.

38. (canceled)

39. The method according to claim 37 wherein said treating additionally comprises at least one of:
i) sweeping said shaft about a pivot point on a surface of said skin layer, said pivot point being defined by said incision;
ii) ensnaring at least a portion of at least one first vein; and
iii) pulling at least a portion of at least one first vein;
iv) puncturing;
v) engaging with at least one tooth;
vi) tearing;
vii removing at least a portion of at least one first vein through said incision; and
viii) severing a first portion of at least one vein from a second portion of at least one vein and leaving said vein in a portion of tissue.

40-41. (canceled)

42. The instrument according to claim 1, wherein said at least two teeth include a plurality of teeth spaced along a forward 2 cm of said shaft.

43. The instrument according to claim 1, wherein said at least two teeth include a plurality of teeth evenly spaced substantially along the entire length of said shaft.
